(19) United States
(12) Patent Application Publication
(10) Pub. No.: US 2002/0068097 A1
(43) Pub. Date: Jun. 6, 2002

(54) Treatment for irritable bowel syndrome and related conditions

(76) Inventor: Amaresh Basu, San Diego, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/861,140

(22) Filed: May 18, 2001

Related U.S. Application Data
(60) Provisional application No. 60/205,851, filed on May 19, 2000.

(51) Int. Cl. A61K 35/84; A61K 35/78

(52) U.S. Cl. 424/725; 424/195.15; 424/735

(57) ABSTRACT

Compositions, including pharmaceutical formulations, of plants and plant material, and specifically, extracts thereof are disclosed for use in the treatment of Irritable Bowel Syndrome, and for use in the treatment related bowel disorders, are disclosed. The compositions of the invention are useful in that they alleviate one or more of the symptoms of Irritable Bowel Syndrome, and of related bowel disorders. A particularly preferred pharmaceutical formulation is disclosed that consists essentially of aqueous extracts of one or more of “Bai Shao” (Paeonia lactiflora), “Bai Zhu” (Atractylodes macrocephala), “Chen Pi” (Citrus reticulata), “Fang Feng” (Saposhnikovia divaricata), together with one or more of “Wu Mei” (Prunus mume), and “Yan Hu” (Corydalis yanhusuo).

### PRIORITY CLAIM

The application claims priority from U.S. patent application Ser. No. 60/205,851, which was filed on May 19, 2000.

### BACKGROUND OF THE INVENTION

### FIELD OF THE INVENTION

The present invention relates to novel formulations of plants and extracts thereof to be used for the treatment of bowel disorders. More specifically, the formulations of the invention can be used to treat conditions such as Irritable Bowel Syndrome (IBS), and other related bowel disorders. The plants have preferably been selected for their ability to address one or more symptomologies associated with IBS, and related bowel disorders.

Irritable Bowel Syndrome

IBS has a reported prevalence generally between 15-25% in both the industrialized and developing world. See Malcolm A, Kellow JE. Irritable Bowel Syndrome. MJA 1998;169:274-279. IBS is characterized, in part, by painful defecation and altered stool frequency/consistency.

IBS, unlike most other diseases of the gastrointestinal tract, is not characterized by any specific, currently known histopathological changes, but rather is a functional disorder characterized, in part, by disturbed gut motility and/or abdominal pain perception linked to cytokines and/or other inflammatory cascades. See Collins SM, Barbara G, Vallance B, Stress, inflammation and the irritable bowel syndrome. Can J Gastroenterol 1999: 13; A:47A-49A; Bueno L, Fioramonti J, “Effects of inflammatory mediators on gut sensitivity” Can J Gastroenterol 1999: 13; A:42A-46A. IBS also occurs in Inflammatory Bowel Disease (IBD) patients who are in remission from their symptoms, see Collins SM, et al. “Putative inflammatory and immunological mechanisms in functional bowel disorders” Baillieres Best Pract Res Clin Gastroenterol 1999: 13; 429-436.

The precise pathophysiology of IBS is not well understood. Nevertheless, there is a heightened sensitivity to visceral pain perception in IBS, known as “peripheral sensitization.” This sensitization involves a reduction in the threshold and an increase in the gain of the transduction processes of primary afferent neurons, attributable to a variety of mediators including monoamines (both catecholamines and indoleamines), substance P, and variety of cytokines and prostanoids including the E-type prostaglandins. See Mayer EA, Gebhart GF: “Basic and clinical aspects of visceral hyperalgesia” Gastroenterology 1994;107:271-293. Also implicated in the etiopathology of IBS is intestinal motor dysfunction (gut dysmotility) which leads to abnormal handling of intraluminal contents and/or gas. See Kellow JE, Phillips SF: “Altered small bowel motility in irritable bowel syndrome is correlated with symptoms” Gastroenterlogy 1987;92:1885-1893; Levitt MD, Fume J, Olsson S: “The relation of passage of gas and abdominal bloating to colonic gas production.” Ann Int Med 1996;124:422-4. Psychological factors may also contribute to IBS symptoms appearing in conjunction with, if not triggered by, disturbances including depression and anxiety. See Drossman DA, Creed FH, Fava GA: “Psychosocial aspects of the functional bowel disorders” Gastroenterlogy Int 1995;8:47-90.

Although the etiology of IBS is not fully characterized, validated diagnostic schemata for IBS are available. For example, the Rome criteria and the Manning criteria allow the diagnosis of IBS to be made based upon patient history. As an example, the Rome criteria requires three months of continuous or recurrent abdominal pain or discomfort that is relieved by defecation and/or associated with a change in stool frequency or consistency as well as two or more of the following: altered stool frequency, altered stool form, altered stool passage, passage of mucus, or bloating and abdominal distention. The absence of any structural or biochemical disorders that could be causing the symptoms is also a necessary condition.

Currently Available Treatments

IBS represents a therapeutic challenge to both clinicians and developers of pharmaceuticals. The uncertainty and variety of causes, as well as the variable nature of symptomatic expression greatly complicates the task of treating this disorder. As noted above, IBS is a functional bowel disorder that is characterized by, for example, abdominal pain and/or discomfort in association with abnormal stool frequency and/or consistency, for example, diarrhea or constipation.

The earliest and most simple treatments have focused on symptomatic relief. For diarrhea-predominant IBS, anti-diarrheal agents such as loperamide and diphenoxylate have been used with some success, especially in acute, situation-specific settings. See Efskind PS, Bernkley T, Vatn MH: “A double blind, placebo-controlled trial with loperamide in irritable bowel syndrome” Scand J Gastroenterol 1996;31:463-8. Dietary supplementation with fiber or psyllium products has typically been recommended to IBS patients, particularly those with constipation-predominant symptoms. More recent studies, however have cast some doubt on the real benefit provided by this strategy. See Lucy MR, Clark ML, Lowndes J, Dawson AM: “Is bran efficacious in irritable bowel syndrome? A double blind, placebo-controlled, crossover study” Gut 1987;28:221-25; Frances CY, Whorwell PJ: “Bran and irritable bowel syndrome: time for reappraisal” Lancet 1994;34:496-500. More aggressive treatments for constipation-predominant symptoms include lactulose, docusate, and prokinetic agents such as cisapride. See Shutze K, Brandstatter G, Dragosics B: “Double-blind study of the effect of cisapride on constipation and abdominal discomfort as components of the irritable bowel syndrome” Aliment Pharmacol Ther 1997;1 1:387-94. Symptomatic relief of the pain associated with IBS has been attempted with a variety of smooth muscle relaxants/antispasmodics as well as anticholinergic agents. Meta-analyses of these studies indicate an efficacy greater than placebo for five agents: cimetropium bromide (antimuscarinic), primaverium/octolinium bromide (calcium antagonists), tremebutine (peripheral opiate antagonist), and mebeverine (anticholinergic). See Klein KB: “Controlled treatment trials in the irritable bowel syndrome” Gastroenterology 1988;95:232-241; Poynard T, Naveu S, Mory B: “Meta-analysis of smooth muscle relaxants in the treatment of irritable bowel” Aliment Pharmacol Ther 1994;8:499-510.

There are a number of prokinetic agents that are currently being examined. Such investigational prokinetic agents include nitric oxide synthase inhibitors, adrenoceptor antagonists, gonadotropin-releasing hormone (GnRH) analogues such as leuprolide, cholecystokinin-a (CCK_{A}) antagonists, and certain opioid receptor antagonists. See DePonti F, Giaroni C, Cosentino M, Lecchini S, Frigo GM: “Adrenergic mechanisms in the control of gastrointestinal motility: from basic science to clinical applications” Pharmacol Ther 1996;69:59-78; Tonini M: “Recent advances in the pharmacology of gastrointestinal prokinetics” Pharmacol Res 1996;33:216-26; Mathias JR, Clench MH, Roberts PH, Reeves-Darby VG: “Effect of leuoprolide acetate in patients with functional bowel disease. Long term follow up after double blind, placebo-controlled study” Dig Dis Sci 1994;39:1155-62; Wettstein JG, Bueno L, Junien JL: “CCK antagonists: pharmacology and therapeutic interest” Pharmacol Ther 1994;62:267-82; Evans DC, Jacobs J, Johnson BG, Cantrell BE, Rothman RB, Schoepp DD, Zimmerman DM, Leander JD, Gidda JS: “LY247636: a selective antagonist for peripheral μ opioid receptors” Gastroenterology 1994;106:A495. Adrenergic β₃ selective agonists are also being examined as potential antispasmodic/smooth muscle relaxant. See DePonti F, Cosentino M, Costa A, Girani M, Gibelli G, D'Angelo L, Frigo GM, Crema A: “Inhibitory effects of SR58611A on canine colonic motility: evidence for a role of β₃-adrenoceptors” Br J Pharmacol 1995;1 14:1447-1453.

Many newer treatments also focus on targets, both peripheral and central, that are implicated in contributing to the cause and progression of IBS. As mentioned above, visceral hyperalgesia (characterized by an abnormally low pain threshold in GI afferent sensory neurons) has been implicated in IBS etiopathology. The role of serotonin (5-HT) and its receptors in the GI tract has also been investigated. While many 5-HT receptor subtypes (5-HT₁, 5-HT₂, 5-HT₃ and 5-HT₄) are known to play a role in enteric neuroregulation and perception, research and product development attention has focused, in particular, on the 5-HT₃ receptor, which is known to be present on substance-P containing afferent neurons within the gut. See Farthing MG: “5-hydroxytriptamine and 5-hydroxytriptamine-3 receptor antagonists” Scand J Gastroenterol 1991;26:92-100; Galligan JJ: “Electrophysiological studies of 5-hydroxytriptamine receptors on enteric neurons” In: Serotonin and Gastrointestinal Function pp109-126, Gaginella TS and Galligan JJ (eds.) CRC Press, Boca Raton. However, while there are several 5-HT₃ antagonists available, including ondansetron (which is an antiemetic) and granisetron, no such antagonist is currently approved by the United States Food and Drug Administration (FDA) for use in treating IBS. (On Feb. 15, 2000, the FDA approved Lotronex (alosetron hydrochloride), a potent serotonin 5HT-₃ antagonist, for use in the treatment for diarrhea-predominant IBS (D-IBS) in women. See Mangel AW, Northcutt AR: “Review article: the safety and efficacy of alosetron, a 5-HT₃ receptor antagonist, in female irritable bowel syndrome patients” Aliment Pharmacol Ther 1999;13 Suppl.2:77-82. However, after notifying the FDA of post-marketing reports that reported serious adverse events associated with Lotronex, Glaxo Wellcome voluntarily withdraw Lotronex from the market.)

Members of the class of 5-HT₄ receptor antagonists has also been studied in this capacity. See Houghton LA, Jackson NA, Whorwell PJ: “5-HT₄ antagonism in irritable bowel syndrome (1BS): effect of SB-207266A on rectal sensitivity and small bowel transit” Gut 1997;4Suppl.3:A26. In addition, other drugs being examined for their modulation of visceral sensitivity include: (i) the opioid κ receptor agonists such as fedotozine, which, unlike agents which act on opioid μ and δ receptors (located in the GI tract), does not appear to have undesirable central effects, and, (ii) the somatostatin analogues such as octreotide. See Junien JL, Riviere P: “The hypersensitive gut-peripheral kappa agonists as a new pharmacological approach” Aliment Pharmacol Ther 1995;9:117-26; Halser WL, Soudah HC, Owyang C: “A somatostatin analogue inhibits afferent pathways mediating perception of rectal distention” Gastroenterology 1993;104:1390-7.

Antidepressants, which have been used for a number of years to treat associated affective disorder in IBS patients, may address the increased pain perception that many of these patients experience. In particular, the tricyclic antidepressants (TCAs), which exert usefulI actions at several locations along the brain-gut axis, may mediate the increased pain perception in these individuals. See Gorard DA, Libby GW, Farthing MJ: “Effect of a tricyclic antidepressant on small intestinal motility in health and diarrhea predominant irritable bowel syndrome” Dig Dis Sci 1995;40:86-95; Peghini P, Katz P, Castell D: “Imiprimine increases pain and sensation thresholds to esophageal balloon distension in humans” Gastroenterology 1997;1 12:A255.

Sigma 1 receptor (“σ1,” “sigma,” or “sigma 1”) agonists have been shown to possess antidepressant and anxiolytic properties. In vitro and in vivo studies have demonstrated that sigma 1 sites are implicated in control of motor behavior, regulation of smooth muscle contraction, and control of gut secretions (specifically alkaline secretions), and therefore may alleviate the increased gut motility and increased gut secretions from which many IBS patients suffer. See Walker J, Matsumoto R, Bowen W, Gans D, Jones K, Walker F: “Control of motor behavior” Neurology 1988; 38; 961-965; Vaupel DB, Shu TP: “Regulation of smooth muscle” Eur J Pharmacol 1987; 139; 125-128; Campbell BG, Bobker DH, Leslie FM, Mefford IN, Weber E: “Regulation of neurotransmitter release” Eur J Pharmacol 1987; 138; 447-449.

The symptomologies of IBS are caused by a complex pharmacology. Current treatment options for IBS and/or related conditions do not adequately address the complexities of IBS and therefore, current treatments remain inadequate or, as exemplified by the recent withdrawal from the United States market of the serotonin 5HT-₃ antagonist Lotronex (alosetron hydrochloride). A treatment that is effective in treating bowel disorders such as IBS, without producing severe adverse effects, is therefore desired. More particularly, a treatment for bowel disorders such as IBS that works via a plurality of pharmacological mechanisms, is desired; such a treatment may be the most efficacious solution to the treatment of IBS. Novel treatment options that may address one or more of the multiple therapeutic targets associated with IBS and related bowel disorders or alleviate one or more of the symptomologies of IBS and the related bowel disorders as described above, are therefore is great demand.

### SUMMARY OF THE INVENTION

One aspect of the invention provides compositions and formulations of plants, or extracts thereof, that are useful in the treatment of IBS, or related bowel disorders. According to another aspect of the invention, these compositions and formulations are useful in the treatment of IBS, or related bowel disorders, because they interact with, and, more specifically, inhibit one or more of the following receptor subtypes: the 5HT_{1A} receptor subtype, interaction with the 5HT₄receptor subtype, interaction with the CGRP receptor, the 5HT₇ receptor subtype, interaction with the dopamine D₁ receptor subtype, interaction with the dopamine D₃ receptor subtype, interaction with the dopamine D₅ receptor subtype, and interaction with the sigma 1 (σ1) receptor subtype. And according to another aspect of the invention, these compositions and formulations are useful in the treatment of IBS, or related bowel disorders, because they inhibit the release of serotonin, the release of dopamine, and./or the release of histamine.

Preferred formulations of the invention consist essentially of one or more plants or plant extracts contained in the traditional formula “Tong Xie Yao Fang”, which is also known as “Painful Diarrhea Essential Formula”, specifically “Bai Shao” (Paeonia lactiflora), “Bai Zhu” (Atractylodes macrocephala), “Chen Pi” (Citrus reticulata), and “Fang Feng” (Saposhnikovia divaricata), together with one or more of the following plants or plant extracts: “Wu Mei” (Prunus mume) and “Yan Hu” (Corydalis yanhusuo). Alternative preferred formulations of the invention consist essentially of pharmaceutically effective amounts of the extract “Bai Shao”, the extract “Bai Zhu”, the extract “Chen Pi”, the extract “Fang Feng”, the extract “Wu Mei”; and either or both of the following extracts: “Yan Hu” or the extract “Wu Mei”. Alternatively, preferred formulations of the invention consist essentially of pharmaceutically effective amounts of the extract “Chen Pi” and either or both of the following extracts: “Yan Hu” or the extract “Wu Mei”.

According to certain preferred embodiments of the invention, formulations comprising either one or both of the extracts “Wu Mei” and “Yan Hu”, possess pharmacological activities that can treat one or more of the symptomologies of IBS not so treated by the constituents of the “Tong Xie Yao Fang” formulation alone. The formulations of the invention treat one or more of the symptomologies of IBS, or other bowel disorders not so treated by Tong Xie Yao Fang, and are preferably administered orally, in daily or twice daily doses to a mammal, including a test mammal such as a mouse or a rat, or to a human, preferably identified as suffering from or likely to suffer from IBS.

In other embodiments, the invention provides methods of treating IBS, and related bowel disorders, through the identification of individuals, or classes of individuals, exhibiting one or more of the symptoms of IBS, and related bowel disorders, and administering compositions and formulations of plants or plant extracts consistent with the compositions of the invention. Numerous means of identifying the symptoms of IBS, and related bowel disorders, and various routes of administration are within the methods of the invention.

### BRIEF DESCRIPTION OF THE DRAWINGS

The accompanying drawings, which are incorporated in and form part of the specification, merely illustrate embodiments of the present invention. Together with the remainder of the specification, they are meant to serve to explain the principles of the invention to those of skill in the art. In the drawings:

FIG. 1 depicts a graphic representation of ligand binding of various plant extracts to serotonin receptors and the inhibition of 5HT_{1A} and 5HT₇ serotonin receptor subtypes. More specifically, FIG. 1(A) graphically depicts the inhibition of ligand binding to the serotonin receptor 5HT_{1A} by the various individual plants from the extract herein designated ANPH 602; and FIG. 1(B) graphically depicts the inhibition of ligand binding to the serotonin receptor 5HT₇ by the various individual plants from the extract herein designated ANPH 602.

FIG. 2 depicts a graphic representation of the dose-response effects of various plant extracts on the inhibition of 5HT₇ serotonin receptor subtype. More specifically, FIG. 2 graphically depicts data used to determine the IC₅₀ values of Yan Hu (FIG. 2A) and of Chen Pi (FIG. 2B) for 5HT₇ receptor ligand binding.

FIG. 3 depicts a graphic representation of the effect of various plant extracts on the inhibition of the 5HT₃ serotonin receptor subtype. More specifically, FIG. 3 graphically depicts, Interaction of individual plants from ANPH 602 with the 5HT₃ receptor.

FIG. 4 depicts a graphic representation of the effect of various plant extracts on the inhibition of the 5HT₄ serotonin receptor subtype. More specifically, FIG. 4 graphically depicts the interaction of individual plants from the extract herein designated ANPH 602 with the 5HT₄ receptor.

FIG. 5 depicts a graphic representation of the effect of various fractions of a Yan Hu extract, on the inhibition of the dopamine D₁ and D₅ receptor subtypes. Specifically, FIG. 5(a) depicts the in vitro binding activity of “Yan Hu” fractions against the dopamine D₁ receptor, and FIG. 5(a) depicts the in vitro binding activity of “Yan Hu” fractions against the dopamine D₅ receptors.

### DETAILED DESCRIPTION OF THE PREFERRED EMBODIMENTS

The present invention provides novel plant extracts, including either the “Yan Hu” (Corydalis yanhusuo) or the “Wu Mei” (Prunus mume) extracts, which, according to one embodiment of the invention, preferably interact with one or more specific biological targets. The specific biological targets with which the various components of formulations or compositions of the invention interact explain the mechanism of action, as these biological targets modulate different aspects, and effect the different symptoms, of the disease. The following biological targets, receptors and receptor subtypes, are most preferred: 5HT_{1A}, 5HT₄, 5HT₇, dopamine D₁, dopamine D₃, dopamine D₅, sigma 1 (σ1), and adrenergic α_{2A} and Histamine H₂ receptor subtypes.

The biological targets also include CGRP receptors. In particular, the CGRP receptors and the serotonin receptors are desirable targets, as are the 5-HT₁ and 5-HT₇ receptors subtypes. Enterochromaffin cells, found in high density in the intestinal mucosa, are a major source of synthesis and storage of serotonin, and express many 5-HT receptor types. These cells are key for regulation of serotonin effects in the gut. As a further example, the histamine H₂ receptor is also a desirable target. The ability of the formulations and compositions of the invention to inhibit this receptor indicates the desirability of the invention, as recent publications indicate that inhibition of gastric acid secretion relieves the symptoms of diarrhea and postprandial urgency that are associated with IBS, functional diarrhea, and related bowel disorders.

Other exemplary, preferred biological targets include the NOS receptors such as the iNOS (inducible nitric oxide synthase) and eNOS (endothelial nitric oxide synthase) receptors, and the VIP1 (vasoactive intestinal peptide 1) receptors, VIP2 (vasoactive intestinal peptide 2) receptors and PACAP (Pituitary Adenylyl Cyclase-Activating Polypeptide) receptors, all of which either produce or control the release of nitric oxide (NO), which plays a role in modulating gut smooth muscle contraction/relaxation. The PACAP (pituitary adenylate cyclase activating protein) receptors are further exemplary targets, as PACAP has been shown to modulate acid secretion in the stomach.

The specific plant extract formulations of the invention may be used alone, or in combination, or with other ingredients, for the treatment of a variety of bowel disorders including IBS. One object of the present invention is to provide novel formulations of plants for the treatment of bowel disorders. Another object of the invention is to provide novel formulations of plants for the treatment of IBS and/or related disorders.

In developing novel remedies for bowel disorders including IBS, according to one embodiment of the invention, herbal preparations and combinations thereof were studied. These preparations included: “Tong Xie Yao Fang” (“Bai Shao” (Paeonia lactiflora), “Bai Zhu” (Atractylodes macrocephala), “Chen Pi” (Citrus reticulata), and “Fang Feng” (Saposhnikovia divaricata)), a preparation of four herbs traditionally used in Chinese medicine for the treatment of IBS-like symptoms. See Zhang, J, “A complete Works of Jingyue”—see page 540 of “Chinese English manual of common-used prescriptions in Traditional Chinese Medicine”, Canton Science and Technology Press, China. Also studied were “Wu Mei” (Prunus mume) and “Yan Hu” (Corydalis yanhusuo). Pharmacological investigations of these herbal components, alone and in various combinations, yielded unexpected results.

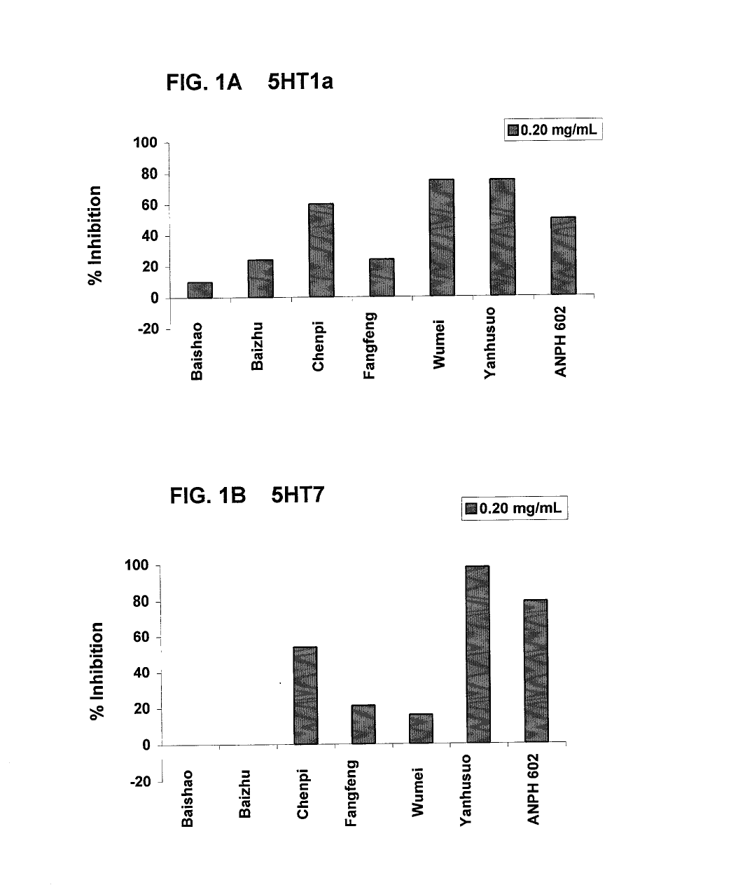

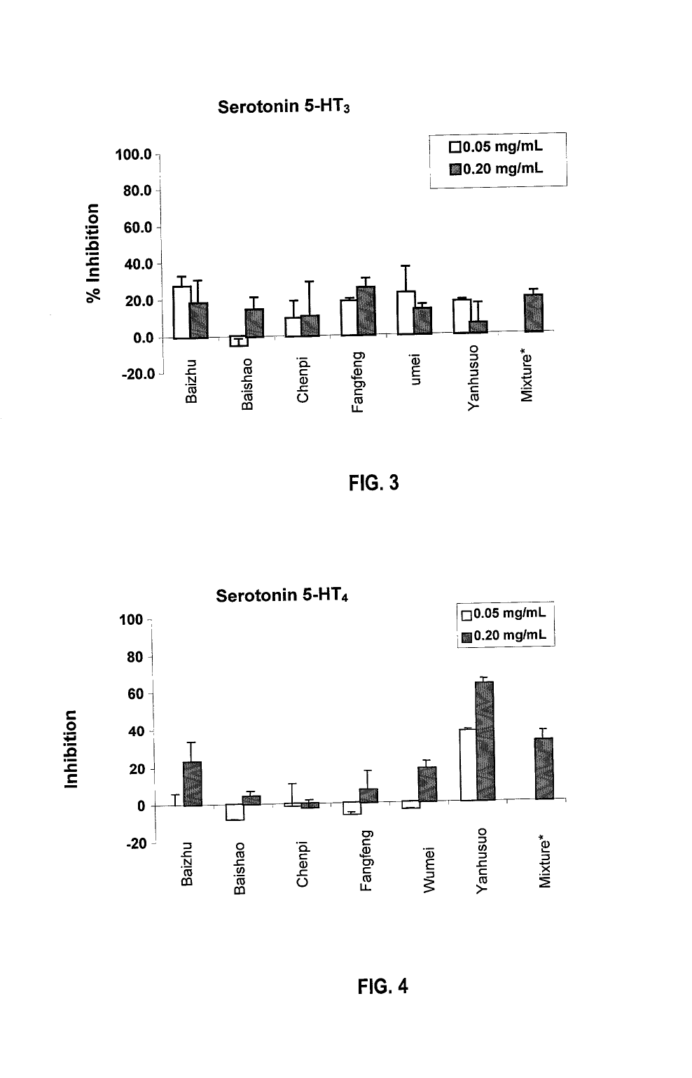

Extracts of these formulations were examined for a wide-range of pharmacological activity (see Tables 3-7 and FIGS. 1-5). “Yan Hu” (Corydalis yanhusuo), “Wu Mei” (Prunus mume), and “Chen Pi” (Citrus reticulata) showed significant activity at the Serotonin 5HT_{1A} receptor. Significant activity against the 5HT₇ receptor subtype is attributable to “Yan Hu” (Corydalis yanhusuo) and, to a lesser degree, to “Chen Pi” (Citrus reticulata) (see FIG. 1). IC₅₀ values were measured for the 5HT₇ subtype “Yan Hu” (Corydalis yanhusuo), and showed a potent IC₅₀ of 5.7 microgram/ml. The IC₅₀ value of “Chen Pi” (Citrus reticulata) was 122 microgram/ml. None of the six herbs significantly interacted with the 5HT₃ receptor subtype (see FIG. 3).

“Yan Hu” (Corydalis yanhusuo), demonstrating a unique pharmacological profile, interacted significantly with the 5HT₄ receptor subtype (see FIG. 4). Even more surprising was the dopamine receptor interactions demonstrated by “Yan Hu” (Corydalis yanhusuo). “Yan Hu” showed more potent activity across separate dopamine receptor subtypes than any of the plants traditionally found in “Tong Xie Yao Fang” (see Table 5). Finally, of the six plants tested, only “Yan Hu” (Corydalis yanhusuo) interacted significantly with the sigma 1 receptor subtype (see Table 6).

In light of the pharmacological benefits outlined above for “Yan Hu” (Corydalis yanhusuo) and “Wu Mei” (Prunus mume), when compared to the activity of the four components typically contained in “Tong Xie Yao Fang,” the invention provides an improved formulation for IBS and/or related disorders using either or both of the two herbs (“Yan Hu” (Corydalis yanhusuo), “Wu Mei” (Prunus mume)) exhibiting said pharmacological benefits, either alone, or in combination, with “Tong Xie Yao Fang.”

Additionally, the pharmacological activity of “Yan Hu” (Corydalis yanhusuo) and/or “Wu Mei” (Prunus mume) indicates that either or both is particularly useful in treatment of IBS and/or related conditions. Their unexpected and significant pharmacological profiles leads to the conclusion that among the many other herbs used for IBS and related conditions, the combinations of herbs herein described may be particularly efficacious for the treatment of lBS and/or related conditions.

Tong Xie Yao Fang

Tong Xie Yao Fang, also known as “Painful Diarrhea Essential Formula”, is a Traditional Chinese Medicine used for certain bowel complaints. It consists of: “Bai Shao” (Paeonia lactiflora), “Bai Zhu” (Atractylodes macrocephala), “Chen Pi” (Citrus reticulata), and “Fang Feng” (Saposhnikovia divaricata). See Zhang, J, “A complete Works of Jingyue”—see page 540 of “Chinese English manual of common-used prescriptions in Traditional Chinese Medicine”, Canton Science and Technology Press, China.

Specific botanicals

“Bai Shao”: Paeonia lactifloria and closely related species

Paeonia lactifloria (which is also known as, especially its root, Paeonia albifloria) exhibits varied activities. Anti-inflammatory activities include inhibition of histamine release from mast cells and inhibition of arachidonic acid metabolism. See Hirai Y, Takase H, Kobayashi H, Yamamoto M, Fujioka N, Kohda H, Yamasaki K, Yasuhara T. Nakajima T: “Screening test for anti-inflammatory crude drugs based on inhibition effect of histamine release from mast cells” Shoyakugaku Zasshi 1983;37:374-80; Umeda M, Amagaya S, Ogihara Y: “Effects of certain herbal medicines on the biotransformation of arachidonic acid: a new pharmacological testing method using serum” J Ethnopharmacol 1988;23:91-8. The inhibition of both leukotriene B4 and oxygen radical generation as well as induction of tumor necrosis factor (TNF) from macrophages has been inhibited in in vitro experiments with these extracts. See Imamichi T, Hayashi K, Nakamura T, Kaneko K, Koyama J: “A Chinese traditional medicine, juzentaihoto, inhibits the 0₂-generation by macrophages” J Pharmacobio Dyn 1989;12:693-699; Li J, Zhao W, Chen M, Xu S: “Effect of total glucosides of Peony on leukotriene B4 formation by peritoneal macrophages in rats” Zhongguo Yaolixue Tongbao 1992;8:36-39; Wang B, Chen M, Xu S: “Effect of total glucosides of Peony (Tgp) on tumor necrosis factor produced by peritoneal macrophages in rats” Zhongguo Yaolixue Tongbao 1995;1 1:36-38. Also seen has been inhibition of Interleukin-1 (IL-1) formation in mouse spleen lymphocytes. See Li J, Liang J, Zhou A, Chen M, Xu X: “Modulatory effects of total glucosides of Peony on B lymphocyte proliferation and interleukin 1 production” Zhongguo Yaolixue Yu Dulixue Zazhi 1994;8:53-55; Kadota S, Basnet P, Terashima S, Li J, Namba T, Kageyu A: “Palbinone, a novel terpenoid from Paeonia albiflora: a potent inhibitory activity on human monocyte interleukin-1beta” Phytother Res 1995;9:375-381. Counted among hepatic effects is modulation of the enzymes glutamate-pyruvate-transaminase, glutamate-oxaloacetate-transaminase, lactate dehydrogenase, and alkaline phosphatase. See Kumazawa N, Ohta S,: Tu S, Kamogawa A, Shinoda M: “Protective effects of various methanol extracts of crude drugs on experimental hepatic injury induced by alpha-naphthylisothiocyanate in rats” Yakugaku Zasshi 1991;111:199-204; Hong N, Bae H, RhoY, Kim N, Kim J: “Studies on the efficacy of combined preparations of crude drug (X1). Effect of Sagan-Tang on the central nervous, cardiovascular system and the liver damage” Korean J Pharmacog 1989;20:196-203.

The inhibition of 3-alpha hydroxy dehydrogenase, an enzyme responsible for steroid metabolism in both the liver and CNS has also been noted. See Kadota S, Terashima, S: Kikuchi T, Namba T: “Palbinone, a potent inhibitor of 3-alpha-hydroxy dehydrogenase, from Paeonia albiflora.” Tetrahedron Lett 1992;33:255-6. Aldose-reductase inhibition has also been reported. See Aida K, Tawata M, Shindo H, Onaya T, Sasaki H, Nishimura H, Chin M, Mitsuhashi H: “The existence of aldose reductase inhibitors in dome kampo medicines (oriental herb prescriptions)” Planta Med 1989;55:22-26; Kadota S, Terashima S, Basnet P, Kikushi T, Namba T: “Palbinone, a novel terpenoid from Paeonia albiflora; potent inhibitory activity on 3a-hydroxysteroid dehydrogenase” Chem Pharm Bull 1993;41:487-90.

DNA polymerase alpha inhibition and RNA reverse transcriptase inhibition have been noted in toxicology studies. See Ono K, Nakane H, Meng Z, Ose Y, Sakai Y, Mizuno M: “Differential inhibitory effects of various herb extracts on the activities of reverse transcriptase and various deoxyribonucleic acid (DNA) polymerases” Chem Pharm Bull 1989;37:1810-2.

“Bai Zhu”: Atractylodes macrocephala and closely related species

Anti-inflammatory activities of this species include inhibition of both 5-Lipoxygenase (5LO) as well as Cyclooxygenase-1 (Cox-1). See Resh M, Steigel A, Chen Zi, Bauer R: “5-lipoxygenase and cyclooxygenase-1 inhibitory active compounds from Atractylodes lancea” J Nat Prod 1998;61:347-50. Additional (potential) anti-inflammatory action as well as activity regulating fluid and electrolyte levels was shown in experiments in both equine and murine renal systems. Specifically, both xanthine oxidase (XO) and the Na-K ATPase were inhibited by extracts of A. macrocephala. See Sakurai T, Yamada H, Saito K, Kano Y: “Enzyme inhibitory activities of acetylene and sesquiterpene compounds in atractylodes rhizome” Bio Pharm Bull 1993;16:142-5; Satoh K, Yasuda I, Nagai F, Ushiyama K, Akiyama K, Kano I: “The effects of crude drugs using diuretic on horse kidney (Na++K+)-adenosine triphosphatase” Yakugaku Zasshi 1991; 1 1 1:1 38-45. In addition to Na-K ATPase, less potent inhibition of the Mg ATPase, H-K ATPase, H ATPase, and Ca ATPase has been reported. See Satoh K, Nagai F, Ushiyama K, Kano I: “Specific inhibition of Na+,K(+)-ATPase activity by atractylon, a major componentof byaku-jutsu, by interaction with enzyme in the E2 state” Biochem Pharmacol 1996;51:339-43. Xanthine oxidase in stomach tissue (involved in pathogenesis of inflammation and ulcer formation) was also inhibited by extracts of this herb. See Sakurai T, Sugawara H, Saito K, Kano Y: “Effects of the acetylene compound from Atractylodes rhizome on experimental gastric ulcers induced by active oxygen species” Bio Pharm Bull 1994; 17:1364-8.

Specific effects on hepatic drug metabolizing enzymes have also been found, including inhibition of lipid peroxidation and of the enzyme species aminopyrine-N-demethylase (APD) and aniline hydroxylase (ANH). See Mayanagi M, Nakayama S, Oguchi K: “Effects of Sino-Japanese herbs in the family Compositae on the hepatic drug metabolizing enzymes and lipid peroxidation in rats” Nippon Yakurigaku Zasshi 1992;100:29-37.

Antioxidant activity has been noted for chemical species found in Atractylodes. See Hwang JM, Tseng TH, Hsieh YS, Chou FP, Wang CJ, Chu CY: “Inhibitory effect of atractylon on tert-butyl hydroperoxide induced DNA damage and hepatic toxicity in rat hepatocytes” Arch Toxicol 1996;70:640-44.

“Chen Pi”: Citrus reticulata and closely related species

As seen for the methylxanthines, C. retiulata extracts exhibit inhibition of cAMP phosphodiesterase and other cyclic nucleotide phosphodiesterases. See Nikaido T, Ohmoto T, Noguchi H, Saitoh H, Sankawa U: “Inhibitors of cyclic AMP phosphodiesterase in medicinal plants” Planta Med 1981 Sep;43(1):18-23; NikaidoT, Ohmoto T, Sankawa U, Hamanaka,T Totsuka K: “Inhibition of cyclic AMP phosphodiesterase by flavonoids” Planta Med 1982;46:162-66. Inhibition of murine aorta Na-K ATPase has been shown to account for the cardiotonic activity seen with these extracts. See Hirai N, Miura T, Moriyasu M, Ichimaru M, Nishiyama Y, Kato A: “Cardiotonic action of the root of Citrus reticulata” Nat Med 97;51:67-8. Hepatic cholesterol metabolism and inhibitory effect on the activities of HMG-CoA reductase and ACAT (cholesterol-O-acyltransferase) are modulated by C. reticulata extracts. See Bok SH, Lee SH, Park YB, Bae KH, Son KH, Jeong TS, Choi MS: “Plasma and hepatic cholesterol and hepatic activities of 3-hydroxy-3-methyl-glutaryl-CoA reductase and acyl CoA: cholesterol transferase are lower in rats fed citrus peel extract or a mixture of citrus bioflavonoids” J Nutr 1999;129:1182-5. Pharmacological studies in anesthetized animals have indicated pressor (vasoconstriction) activity; such activity is mediated, in part, by alpha adrenergic stimulation as well as by the inhibition of histamine-induced bronchoconstriction. See Yao S, Wang L, Yeung S: “Yanhusuo” Pharmacology and applications of Chinese Materia Medica 1986:682-4. Antioxidant activity has been documented as has antimicrobial and antimutagenic activity. See Tanizawa H, Ohkawa Y, Takino Y, Miyase T, Ueno A, Kageyama T, Hara S: “Studies on natural antioxidants in citrus species. I. Determination of antioxidative activities of citrus fruits” Chem Pharm Bull 1992;40:1940-2.

“Fang Feng”: Saposhnikovia divaricata and closely related species

S. divaricata inhibits the expression of inducible nitric oxide synthase (iNOS) (and thus limit NO generation) in macrophage cell lines. See Wang CC, Chen LG, Yang L: “Inducible nitric oxide synthase inhibitor of the Chinese herb I. Saposhnikovia divaricata (Turcz.) Schischk” Cancer Lett 1997;145:151-7. Also in macrophages, phagocytic activity is increased by Saposhnikovia extracts as shown in in vivo carbon clearance tests. See Shimizu N, Tomada M, Gonda R, Kanari M, Kubota A, Kubota A: “An acidic polysaccharide having activity on the reticuloendothelial system from the roots and rhizomes of Saposhnikovia divaricata” Chem Pharm Bull 1989;37:3054-7.

“Yan Hu”: Corydalis vanhusuo and closely related species

C. yanhusuo and closely related species (which contain many of the same active alkaloid constituents such as protopine) show a variety of activities in both the central and peripheral nervous system including postsynaptic neuromuscular blockade (for example, nondepolarizing) and inhibition of acetylcholinesterase. See Yao S, Wang L, Yeung S: “Yanhusuo” Pharmacology and applications of Chinese Materia Medica 1986:515-24; Kim SR, Hwang SY, Jang YP, Park MJ, Markelonis GJ, Oh TH, Kim YC: “Protopine from Corydalis ternata has anticholinesterase and antiamnesic activities” Planta Med 1999;65:218-21. CNS effects span a broad range, and include antagonism of the dopamine D₂ receptor and/or the serotonin 5-HT₂ receptor, leading to hypotension and bradycardia in rats. See Lin MT, Chueh FY, Hsieh MT, Chen CF: “Antihypertensive effects of DL-tetrahydropalmatine: an active principle isolated from Corydalis” Clin Exp Pharmacol Physiol 1996;23:738-42. The substance isocoryne, isolated from C. pseudoadunca, has been shown to affect the GABA receptor complex in an inhibitory fashion. See Chemevskaja NI, Krishtal OA, Valeyev AY: “Inhibitions of the GABA-induced currents of rat neurons by the alkaloid isocoryne from the plant Corydalis psuedoadunca” Toxicon 1990;28:727-30. The related species C. cava has been shown to have activity in inhibiting the dimerization (and potentiating the activity) of the endogenous analgesic pentapeptides met- and leu-enkephalin as well as enhancing the chemical oxidation of epinephrine. See Kleber E, Schneider W, Schafer HL, Elstner EF: “Modulation of key reactions of the catecholamine metabolism by extracts from Eschscholtzia californica and Corydalis cava” Arzneimittelforschung 1995 Feb;45(2):127-31; Reimeier C, Schneider I, Schneider W, Schafer HL, Elstner EF: “Effects of ethanolic extracts from Eschscholtzia californica and Corydalis cava on dimerization and oxidation of enkephalins” Arzneimittelforschung 1995 Feb;45(2):132-6. C. cava, and the protopine alkaloids found in many Corydalis species including C. yanhusuo, have been found to interact with the GABA-receptor complex in a fashion comparable to the benzodiazepines. See Reichert: “Sedative Effects of California Poppy and Corydalis” Quarterly Review of Natural Medicine 1996;Winter:256.

Other activities of these extracts, and the protopine species isolated therefrom, include inhibition of platelet aggregation, decrease of intracellular calcium, and inhibition of thromboxane formation/phosphoinositide species breakdown. See Ko FN, et al.: “Antiplatelet effects of protopine isolated from Corydalis tubers” Thromb Res 1989;56:289-98; Matsuda H, Shiomoto H, Namba K, Kubo M: “Effects of protopine on blood platelet aggregation” Planta Medica 1988:498-501. Both voltage and adrenoceptor-gated calcium influx in rat aorta was blocked by protopines. See Ko FN, Wu TS, Lu ST, Wu YC, Huang TF, Teng CM: “Ca(2+)-channel blockade in rat thoracic aorta by protopine isolated from Corydalis tubers” Jpn J Pharmacology 1992;58:1-9.

In experiments examining cataract formation in laboratory rats, Corydalis extract was shown to reduce the activity of aldose reductase. See Kubo M, Matsuda H, Tokuoka K, Kobayashi Y, Shiping M, Tanaka T: “Studies of anti-cataract drugs from natural sources. I. Effects of a methanolic extract and the alkaloidal components from corydalis tuber on in vitro aldose reductase activity” Bio Pharm Bull 1994;17:458-9.

Finally, anti-inflammatory activity including suppression of histamine release from mast cells has been noted. See Kubo M, Matsuda H, Tokuoka K, Shiping M: “Anti-inflammatory activities of methanolic extract and alkaloidal components from Corydalis Tuber” Bio Pharm Bull 1994;17:262-5.

“Wu Mei”: Prunus mume and closely related species

Antibacterial, antihelminthic, and antimutagenic activity have been reported for this plant and its extracts. Calcium antagonism and vasodilatory activity has been found using murine aorta. See Ichikawa K., Kinoshita T., Sankawa U: “The screening of Chinese crude drugs for Ca+2 antagonist activity: identification of active principles from the aerial part of Pogostemon cablin and the fruits of Prunus mume” Chem Pharm Bull 1989;37:345-48. Weak activities have been found for both platelet activating factor binding inhibition as well as tyrosinase, which is involved in melanin synthesis, inhibition. See Han B, Yang O, KimY, Han Y: “Screening of the platelet activating factor (PAF) antagonistic activities on herbal medicines” Yakhak Hoe Chi 1994;38:462-8; Matsuda H, Nakamura S, Kubo M: “Studies of cuticle drugs from natural sources. Ii. Inhibitory effects of prunus plants on melanin biosynthesis” Biol Pharm Bull 1194;17:1417-20.

The extract or extracts produced according to the methods of the invention may be administered, in formulations of the invention, to an individual in a dose containing a pharmaceutically-effective amount of the extract or extracts, or components thereof. This administration can be through any effective route. It is contemplated that administration may be effected, for example, and preferably orally, and may also be effected intramuscularly, subcutaneously, intraperitoneally, transdermally, transmucosally, buccally, or through inhalation or pulmonary infusion. Dosages that are contemplated for a 70 kg adult human range from a lower limit of 10, 25, 50, 100, 150, 200, or 250 mg to an upper limit of 750, 1000, 1500, 2000, 2500, 3000, 4000, 5000, or up to 10,000 mg of the extracts used in Examples 1 and 2, or other extracts. Dosages may also be calculated on the basis of mg per surface area of subject. Preferred dosages for a 70 kg human are from about 200, 500, or 750 mg to about 2000, 3000, 4000, or 5000 mg. These dosages can be administered once, twice or up to four times per day, or two or more dosages may be combined into a controlled release formulation or dermal or mucosal patch or subdermal administration mechanism of known type or may be administered through an infusion device over a period of time. The dose may also be tailored to achieve one or more a desired effects, and will depend on such factors as weight, diet, concurrent medication and other factors that those skilled in the medical arts will recognize.

The present invention also encompasses pharmaceutical compositions comprising a pharmaceutically acceptable carrier prepared for storage and subsequent administration, which have a pharmaceutically effective amount of the extract disclosed above in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985). Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used.

These compositions may be formulated and used as instant teas, teas, tablets, capsules or liquid elixirs for oral administration; suppositories for rectal administration; sterile solutions, suspensions for injectable administration; or other formulations known in the art. Suitable excipients are known in the art and include, for example, water, saline, dextrose, mannitol, lactose, lecithin, albumin, sodium glutamate, cysteine hydrochloride, and the like. In addition, if desired, the pharmaceutical compositions may contain relatively small amounts of nontoxic auxiliary substances, such as wetting agents, pH buffering agents, and the like, as are known to persons of skill in the art.

In practicing the compositions of the invention, the formulated dosage may be used alone or in combination with other therapeutic or diagnostic agents. These products can be utilized in vivo, ordinarily in a mammal, preferably in a human, or in vitro. In employing them in vivo, the products or compositions can be administered to the mammal in any of a variety of manners known to persons skilled in the art, including parenterally, intravenously, subcutaneously, intramuscularly, colonically, rectally, nasally or intraperitoneally, and may employ any of a variety of dosage forms.

As used herein, the term “syndrome”, “disorder”, or “disease” refer, either individually or collectively, to any syndrome, disorder, disease, condition, or combination of manifestations or symptoms recognized or diagnosed as a syndrome, disorder, or disease. If modified by a phrase such as bowel or inflammatory or irritable or modified by one or more or a set of manifestations or symptoms, that usage of “syndrome” refers to any such syndrome, disorder, disease, condition, or combination of such manifestations or symptoms recognized or diagnosed as a such syndrome.

As used herein, the term “pharmaceutically effective amount” refers to an amount sufficient to alleviate, in any degree or manner, one or more of the manifestations or symptoms recognized or diagnosed as associated with the modifying syndrome or disorder, the modifying manifestations, or the modifying symptom.

As used herein, the term “bowel disorder” refers to any recognized disorders functionally or symptomatically related to Irritable Bowel Syndrome (IBS), including, for example, Irritable Bowel Disease (IBD), the following Functional Bowel Disorders (FBDs): Functional Constipation, Functional Diarrhea, Chronic or Non-chronic Functional Abdominal Pain, and Functional Abdominal Bloating; the following disorders having symptoms generally localized in the stomach and/or esophagus: Functional Dyspepsia (for example, Ulcer-like Dyspepsia and Dysmotility-like Dyspepsia), Functional Heartburn, Non-ulcer Dyspepsia, Non-cardiac Chest Pain, and Functional Chest Pain of Presumed Esophageal Origin; the following disorder that is particularly diagnosed in women: Chronic Pelvic Pain; and the following disorder that is particularly diagnosed in children: Recurrent Abdominal Pain (RAP).

As used herein, the term “plant” or “plant material” includes, most preferably, the root or Rhizoma of the plant, the Radix, the Pericarpium, and the Fructus of the designated plant or closely related plant, as defined herein.

As used herein, the specific disclosed plant species, for example, “Bai Shao” (Paeonia lactiflora), “Bai Zhu” (Atractylodes macrocephala), “Chen Pi” (Citrus reticulata), “Fang Feng” (Saposhnikovia divaricata), “Wu Mei” (Prunus mume), and “Yan Hu” (Corydalis yanhusuo), include the particularly described species and any “closely related species” of plant. Such closely related species may be identified and characterized by those of skill in the art on the basis of their phenotypical similarity to the particularly described species or on the basis of any accepted botanical classification system or, most preferably, may be identified and characterized by those of skill in the art on the basis of the similarity of the physical chemical profile of their extracts, as may be characterized by any of a variety of known techniques, such as High Performance Liquid Chromatography (HPLC) and Nuclear Magnetic Resonance Spectroscopy (NMR).

Irritable Bowel Syndrome (IBS) is a complex, heterogeneous disorder characterized by a variety of symptoms that include abdominal pain, constipation and diarrhea. Patients with IBS have alterations in colonic motility and an increased sensitivity to bowel and rectal wall distension, also known as visceral hyperalgesia, and dyspepsia. IBS patients also have frequent abnormalities on psychological testing and are more likely to attribute the symptoms to stress. Several mechanisms have been proposed to be involved in the pathophysiology of IBS. Visceral hypersensitivity is currently the most widely accepted mechanism responsible for both abnormalities in gastrointestinal motility and abdominal pain. Over the years numerous pharmacological pathways have been targeted to understand the disease and to develop targeted therapy.

Since there are numerous possible targets for IBS, the extract herein designated ANPH 602 was tested against a panel of 82 receptor subtypes. Table 2 summarizes the targets with which ANPH 602 extract interact with high affinity. Summarized below is a discussion on each of these targets and the link to the etiology of IBS.

Dopaminergic system

Specific dopamine D₂ antagonists are currently being investigated for the treatment of IBS. However, ANPH 602 showed the following order of potency against the dopamine receptor sub-types: D₅>D₁>D₃>D_{2L}. There are two sub-families of Dopamine receptors (D₁-like receptors that include D₁ and D₅; and D₂-like receptors that include D₂, D₃, and D₄). ANPH 602 seems to have a greater affinity for the D₁-like receptors. Most of the activity of ANPH 602 on Dopamine receptors can be attributed to the plant Yan Hu and the results indicate that the active compound(s) can be extracted either by water or 70% ethanol. Dopamine is a protective agent in the GI tract and Dopamine D₅ receptors have been found in significant amounts in both gastric and duodenal mucosa. Investigators have speculated that D₅ receptors may serve as a target for locally produced dopamine in the GI tract. Dopamine D₃ receptor agonists have been shown to reduce gastric acid, pepsin secretion and experimental gastric mucosal injury in rats.

GABA-ergic system

GABA is an inhibitory neurotransmitter and is speculated to play an integral role in brain-gut interactions. GABA-A receptors in the dorsal vagal complex produce marked gastric motor excitation. Orally administered GABA and GABA agonists have been shown to be ulcer protective. In the CNS, GABA-A receptor modulation may play a beneficial role in controlling the stress component of IBS.

Glutamate receptors

Glutamate is an excitatory neurotransmitter and like GABA is speculated to play an integral role in brain-gut interactions. Both kainic acid and NMDA have been shown to produce dose-related increases in intragastric pressure and motility. In addition, glutamate receptors have been implicated in the modulation of visceral hyperalgesia.

Histamine receptors

H₂ receptor antagonists (e.g. Tagamet, Zantac and Pepcid) have been used for several decades as potent inhibitors of gastric acid secretion (and its H+ content). Patients with gastroesophageal reflux disease (GERD), who also have chronic diarrhea and postprandial urgency due to IBS, show rapid and marked improvement when treated with specific H₂ blockers. Thus, inhibition of gastric secretion, by blocking H₂ receptors, may effectively control some of the symptoms of IBS.

Calcitonin Gene-Related Peptide (CGRP)

CGRP is a sensory neuropeptide known to be involved in visceral hyperalgesia. In various animal models CGRP has been found to control intestinal motility and inhibit gastric emptying. CGRP receptor antagonists have been shown to reverse the sensitizing effects of acetic acid on nociceptive response to colorectal distension.

Purinergic P_{2Y}

Adenosine is a neuromediator that exhibits interesting antinociceptive properties. Purinergic receptors of the A₂ type (P_{2Y}) are present on terminal endings of visceral primary afferents. These receptors have also been shown to be involved in the regulation of intestinal relaxation and contraction, which are thought to be mediated through the modulation of nitric oxide (NO) synthetase.

Serotonergic receptors

Antagonists of Serotonin 5-HT₃ and 5-HT₄ receptors are currently being investigated for the treatment of IBS. However, ANPH 602 appears to strongly inhibit binding to 5-HT₇ and 5-HT₁ receptors. Studies with individual plants indicated that none of them interact with 5-HT₃, while only Yan Hu interacted with 5HT₄ receptor. Yan Hu, Wu Mei and Chen Pi contribute towards the interaction of ANPH 602 with 5-HT_{1A}, while Yan Hu and, to a lesser extent, Chen Pi contribute towards 5-HT₇ interaction. 5-HT_{1A} receptor blockers abolish the signs of abdominal pain related to duodenal distension, suggesting that 5-HT₁ receptor subtypes are involved in the modulation of sensory pathways from the gut. A recent study has shown that 5-HT₇ receptors modulate contraction and relaxation of human colonic circular smooth muscle and thus control gut motility.

Sigma receptors

These receptors are known to be involved in both the regulation of gut motility and gut secretions (specifically alkaline secretions). In addition, sigma agonists have shown anti-depressant and anxiolytic properties and may be beneficial in modulating the stress component of IBS.

Adrenergic α_{2A}

Activation/inhibition of adrenergic receptors would effect sympathetic regulation of gut finction. Idazoxan (an α₂-receptor antagonist) has been investigated as a possible prokinetic agent for the treatment of IBS while clonidine (and α₂-receptor agonist) has been shown to have strong antinociceptive activity.

Conclusion

In summary, there is ample support to suggest that etiology of IBS is multi-factorial. Therefore, treatment regimens, targeting only specific pharmacological pathways, will not benefit all the diverse symptoms of IBS. The present inventors have shown that specific herbs contained within ANPH 602, but not contained within Tong Xie Yao Fang, interact with several different pharmacological targets involved in the symptoms of IBS and/or related bowel conditions. Based on these observations, it is believed that the ANPH 602 recipe will be a superior therapeutic agent, when compared with Tong Xie Yao Fang and like recipes, for the treatment of various symptoms of IBS and/or related conditions.

The various articles of the scientific and/or medical literature and texts cited herein are hereby incorporated by reference; each constitutes a part of the disclosure of this specification. Furthermore, while specific embodiments, and working and prophetic examples of the invention have been described in detail to illustrate the broad applicability and principles underlying the invention, it will be understood by those of skill in the art that the invention may be embodied otherwise without departing from such broad applicability and principles.

### EXAMPLES

The following examples are meant to illustrate specific, preferred embodiments of the invention, and are not meant to limit the scope of protection afforded by the invention.

### Example 1

### IN VITRO ASSAYS

Extracts of the plant species described above were produced and tested in in vitro assay systems indicative of certain of the above-described symptomologies that have been associated with bowel disorders, including IBS and IBD.

Materials

Individual herbs were purchased from various traditional Chinese Medicine pharmacies in the People's Republic of China, Shanghai. While the quality of the herbs may vary slightly among the various commercial sources, the inventors have no preferred source from among traditional Chinese Medicine pharmacies in the People's Republic of China, Shanghai. The herbs were combined and extracted by methods familiar to those skilled in the art and according to the proprietary formula generated by Ancile Pharmaceuticals.

The herbs and the parts of the plant used to prepare ANPH 602 extract are described below in Table 1:

Plant Part Utilized

TABLE 1
Composition of extract herein designated: ANPH 602.

| Plant | Part | Latin-Name |
|---|---|---|
| Bai Zhu | Rhizoma | Atractylodes macrocephala Koidz. |
| Bai Shao | Radix | Paeonia lactiflora Pall. |
| Chen Pi | Pericarpium | Citrus reticulata Blanco Var. Unshin |
| | | (Marcon) H. H. Hu |
| Fang Feng | Radix | Saposhnikovia divaricata (Turcz.) Schischk. |
| Wu Mei | Fructus | Prunus mume (Sieb.) sieb et Zucc. |
| Yan Hu | Rhizoma | Corydalis yanhusuo W. T. Wang |

Extracts were also prepared from the individual plants that comprise the ANPH 602 extract. Two different extraction procedures were used, one by boiling with water and the second by extraction with 70% ethanol. The description of the individual plant extracts are summarized below in Table 2:

TABLE 2
Description of Extracts prepared from individual plants of ANPH 602.

| Plant | Extract Code | Remarks |
|---|---|---|
| ANPH 602 | MH-018-53-A-fd | Water Extract |
| | MH-018-53-B-fd | 70% Ethanol Extract |
| Chen Pi | MH-018-54-A-fd | Water Extract |
| | MH-018-54-B-fd | 70% Ethanol Extract |
| Fang Feng | MH-018-55-A-fd | Water Extract |
| | MH-018-55-B-fd | 70% Ethanol Extract |
| Bai Shao | MH-018-56-A-fd | Water Extract |
| | MH-018-56-B-fd | 70% Ethanol Extract |
| Bai Zhu | MH-018-57-A-fd | Water Extract |
| | MH-018-57-B-fd | 70% Ethanol Extract |
| Yan Hu | MH-018-58-A-fd | Water Extract |
| | MH-018-58-B-fd | 70% Ethanol Extract |
| Wu Mei | MH-018-59-A-fd | Water Extract |
| | MH-018-59-B-fd | 70% Ethanol Extract |

Methods

Sample preparation: Test samples were prepared in water. Prior to analysis, the stock solutions were diluted in appropriate buffer for each receptor binding assay.

Assays

Assays were performed at the following analytical laboratories:

Panlabs Taiwan, Ltd. 158 Li-Teh Road, Peitou Taipei, Taiwan R.O.C.

Ancile Pharmaceuticals 10555 Science Center Drive San Diego, Calif. 92121

The list of the assays comprising the initial screen is given in Table 3. ANPH 602 extract was tested at a concentration of 200 μg/ml in duplicate. Individual plant extracts were tested against Dopamine D₁, D₃ & D₅ and 5HT_{1A} & 5HT₇ receptors at various concentrations, which were developed in house. A brief description of the assays are provided. For IC₅₀ calculations, Graphpad-Prizm version 3 software was used.

Results

The extract ANPH 602 was profiled against 82 separate, major receptor subtypes. The results are outlined in Table 3. The results indicate that ANPH 602, at a concentration of 200 μg/ml, interacts with several targets with varying affinities. ANPH 602 interacts with high affinity based on an inhibition of ligand binding of approximately 50% or more, to nine classes of receptors. That list is provided in Table 4.

TABLE 3
Inhibition of Ligand Binding by ANPH 602 Water Extract
(200 μg/mL) in Several In Vitro Receptor Ligand Binding Assays
(N = 2)

| | | % |
|---|---|---|
| Assay | Source | Inhibition |
| Adenosine A₁ | human | 8 |
| Adenosine A_{2A} | human | 15 |
| Adenosine A_{2B} | human | 9 |
| Adrenergic α_{1A} | rat | 25 |
| Adrenergic α_{1B} | rat | 31 |
| Adrenergic α_{1D} | human | 43 |
| Adrenergic α_{2A} | human | 76 |
| Adrenergic α_{2B} | human | 30 |
| Adrenergic α_{2C} | human | 30 |
| Adrenergic β₁ | human | 28 |
| Adrenergic β₂ | human | 19 |
| Adrenergic β₃ | human | 27 |
| Angiotensin AT₁ | human | −9 |
| Angiotensin AT₂ | human | 21 |
| Bombesin | rat | 28 |
| Bradykinin B₂ | Guinea pig | 9 |
| Calcitonin Gene-Related Peptide (CGRP) | human | 49 |
| Calcium Channel Type L, | rat | 12 |
| Benzothiazepine | | |
| Calcium Channel Type L, | rat | 3 |
| Dihydropyridine | | |
| Calcium Channel Type L, | rat | 36 |
| Phenylalkylamine | | |
| Calcium Channel Type N | rat | 18 |
| Cannabinoid CB₁ | human | −1 |
| Cannabinoid CB₂ | human | −2 |
| Cholecystokinin CCK_{A} | human | 10 |
| Cholecystokinin CCK_{B} | human | 19 |
| Dopamine D₁ | human | 71 |
| Dopamine D_{2L} | human | 33 |
| Dopamine D₃ | human | 55 |
| Dopamine D_{4.2} | human | 21 |
| Dopamine D₅ | human | 77 |
| Endothelin ET_{A} | human | −26 |
| Endothelin ET_{B} | human | 25 |
| Estrogen ER_{α} | human | 8 |
| GABA_{A, Agonism Site} | rat | 95 |
| GABA_{A, Benzodiazepine, Central} | rat | 36 |
| GABA_{A, Chloride Channel, TBOB} | rat | 43 |
| Galanin | human | 5 |
| Glucocorticoid | human | 8 |
| Glutamate, Kainate | rat | 63 |
| Glutamate, NMDA, Agonist | rat | 92 |
| Glycine, Strychnine-Sensitive | rat | 19 |
| Histamine H₁, Central | Guinea pig | 1 |
| Histamine H₂ | Guinea pig | 80 |
| Histamine H₃ | rat | 33 |
| Interleukin IL-1α | mouse | 7 |
| Leukotrine D₄ | Guinea pig | 8 |
| Muscarinic M₁ | human | 2 |
| Muscarinic M₂ | human | −5 |
| Muscarinic M₃ | human | 2 |
| Muscarinic M₄ | human | 0 |
| Muscarinic M₅ | human | 13 |
| Neuropeptide Y₁ | human | 16 |
| Neuropeptide Y₂ | human | 29 |
| Nicotinic Acetylcholine, Central | rat | 3 |
| Opiate δ | human | −6 |
| Opiate κ | human | 29 |
| Opiate μ | human | 20 |
| Orphanin ORL₁ | human | 33 |
| Potassium Channel [K_{V}] | rat | −3 |
| Potassium Channel [SK_{Ca}] | rat | 33 |
| Purinergic P_{2X} | rabbit | 32 |
| Purinergic P_{2Y} | rat | 54 |
| Serotonin 5-HT_{1A} | human | 52 |
| Serotonin 5-HT_{1B} | human | 56 |
| Serotonin 5-HT_{1D} | human | 64 |
| Serotonin 5-HT₂ | rat | 33 |
| Serotonin 5-HT_{2A} | human | 20 |
| Serotonin 5-HT₃ | human | 19 |
| Serotonin 5-HT₄ | Guinea pig | 32 |
| Serotonin 5-HT_{5A} | human | 0 |
| Serotonin 5-HT₆ | human | 27 |
| Serotonin 5-HT₇ | human | 72 |
| Sigma σ₁ | Guinea pig | 94 |
| Sigma σ₂ | rat | 25 |
| Tachykinin NK₁ | human | 27 |
| Tachykinin NK₂ | human | 4 |
| Testosterone | rat | 31 |
| Thromboxane A₂ (TxA₂) | rabbit | 22 |
| Tumor Necrosis Factor (TNF), Non-Selective | human | 3 |
| Vascular Endothelial Growth Factor (VEGF) | human | 23 |
| Vasoactive Intestinal Peptide VIP₁ | human | −23 |
| Vasopressin V_{1A} | human | 33 |

TABLE 4
Receptor sub-types with inhibition of ligand binding of equal to or
greater than approximately 50% by 200 μg/ml of ANPH 602.

| Assay | Source | % Inhibition |
|---|---|---|
| Adrenergic α_{2A} | human | 76 |
| Calcitonin Gene-Related Peptide (CGRP) | human | 49 |
| Dopamine D₁ | human | 71 |
| Dopamine D₃ | human | 55 |
| Dopamine D₅ | human | 77 |
| GABA_{A}, Agonism Site | rat | 95 |
| Glutamate, Kainate | rat | 63 |
| Glutamate, NMDA, Agonist | rat | 92 |
| Histamine H₂ | Guinea pig | 80 |
| Purinergic P_{2Y} | rat | 54 |
| Serotonin 5-HT_{1A} | human | 52 |
| Serotonin 5-HT_{1B} | human | 56 |
| Serotonin 5-HT_{1D} | human | 64 |
| Serotonin 5-HT₇ | human | 72 |
| Sigma σ₁ | Guinea pig | 94 |

Effect of the individual plant components of ANPH 602 Composition

To understand the role of individual plants of ANPH 602, extracts were prepared from each plant and tested against some of the relevant targets in which ANPH 602 showed significant interaction (See Table 4). The effects of individual plant extracts on Serotonin, Dopamine and Sigma receptors are summarized below.

Interaction with Serotonin Receptors

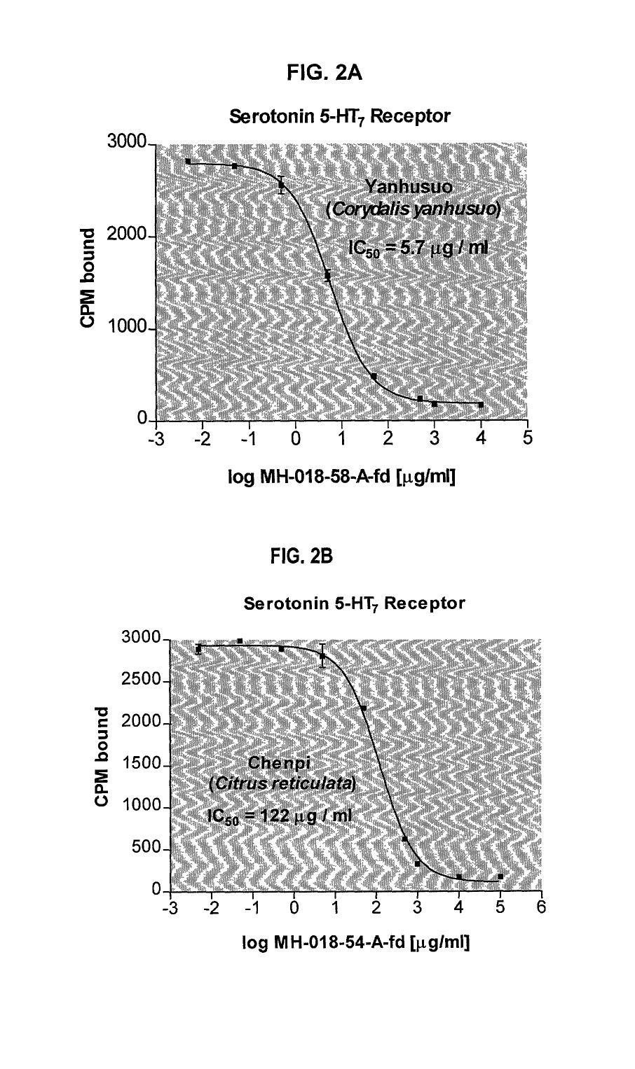

The water extract of ANPH 602 (MH-018-53-A-fd) and the water extracts of the six herb components of ANPH 602 (MH-018-54-A-fd to MH-018-59-A-fd) were tested against the serotonin 5-HT_{1A} and the serotonin 5-HT₇ receptor at a concentration of 200 μg/ml. The ANPH 602 composition inhibits radio-ligand binding to the 5-HT_{1A} receptor by 50% and to the 5-HT₇ receptor by 79% (see FIG. 1). Three plant components of ANPH 602 are mainly responsible for the activity against the 5-HT_{1A} receptor: Yan Hu (Corydalis yanhusuo, 75%), Wu Mei (Prunus mume, 75%) and Chen Pi (Citrus reticulata, 60%). The remaining three herbs showed very low activity. All the individual extracts were tested at a concentration of 200 μg/ml. The activity against the 5-HT₇ receptor can mainly be attributed to Yan Hu (Corydalis yanhusuo) and, to a lesser extent, Chen Pi (Citrus reticulata). The water extract of Yan Hu inhibits radioligand binding to the 5-HT₇ receptor by 98% at 200 μg/ml and has an IC₅₀ value of 5.7 μg/ml. At the same concentration, Chen Pi inhibits binding to the 5-HT₇ receptor by 54% and has an IC₅₀ value of 122 μg/ml (FIG. 2).

Initial screening (Table 3) indicated that ANPH 602 extract has minimal interaction with 5HT₃ and 5HT₄ receptors. A pilot study was conducted to determine if extracts of individual plants behaved differently with these receptors. The results revealed that none of the plants showed any significant interaction with the 5HT₃ receptor (FIG. 3) at testing concentrations of 50 and 200 μg/ml, while only Yan Hu showed significant interaction (˜60% inhibition at 200 μg/ml) with the 5HT₄ receptor (FIG. 4).

Interaction with Dopamine receptors

Effects of individual plant extracts on ligand binding to dopamine receptors D₁, D₃ and D₅ are shown in Table 5. The results clearly indicate that only the extract of Yan Hu is responsible for the interaction of ANPH 602 with dopamine receptors. Hydroalcoholic extracts made from ANPH 602 and Yan Hu were also tested for their ability to inhibit ligand binding with these receptors and the results indicate that the active components can be extracted either by water or 70% ethanol.

TABLE 5
Interaction of ANPH 602 and individual plant extracts with
dopamine receptors

| | | Receptor (% Inhibition) | | |
|---|---|---|---|---|
| | | Dopamine | Dopamine | Dopamine |
| Plant | Code | D₁ | D₃ | D₅ |
| ANPH 602 | MH-018-53-A-fd | 76 | 21 | 83 |
| | (water extract) | | | |
| | MH-018-53-B-fd | 74 | NT | 84 |
| | (ethanol extract) | | | |
| Chen Pi | MH-018-54-A-fd | 22 | 25 | 24 |
| | (water extract) | | | |
| Fang Feng | MH-018-55-A-fd | 20 | 27 | 30 |
| | (water extract) | | | |
| Bai Shao | MH-018-56-A-fd | 22 | 20 | 26 |
| | (water extract) | | | |
| Bai Zhu | MH-018-57-A-fd | 30 | 13 | 24 |
| | (water extract) | | | |
| Yan Hu | MH-018-58-A-fd | 96 | 71 | 99 |
| | (water extract) | | | |
| | MH-018-58-B-fd | 98 | 89 | 100 |
| | (Ethanol extract) | | | |
| Wu Mei | MH-018-59-A-fd | 38 | 23 | 37 |
| | (water extract) | | | |

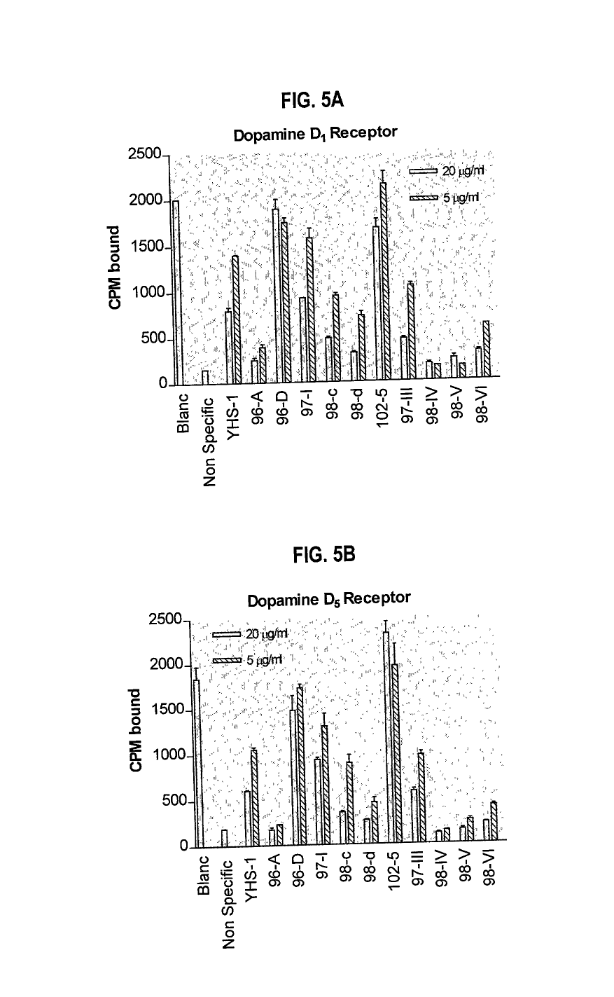

Further experiments on the water extract of Yan Hu were performed. Extracts were adjusted to pH 6 and re-extracted with dichloromethane. The activity found against the dopamine D₁ and D₅ receptors was enriched in the dichloromethane fraction (data not shown). Further separation of the dichloromethane fraction via silica gel chromatography yielded 6 fractions. The activity was further enriched in fractions KY-016-98-IV and KY-016-98-V (FIG. 5) [as indicated by the drop in radioactivity bound to the receptors]. The major component of fraction KY-016-98-IV was found to be isocorypalmine. The predominant compounds in fraction KY-016-98-V are characterized by molecular weights of m/z=372, 335 and 336.

Interaction with Sigma 1 receptor

Effect of water extract of ANPH602 and of individual plant extracts on ligand binding to the sigma 1 receptor are shown in Table 6. The results clearly indicate that only the extract of Yan Hu is responsible for the interaction of ANPH 602 with the sigma 1 receptor.

TABLE 6
Interaction of ANPH 602 and individual plant extracts with the
sigma 1 receptor.

| Extract | Lot No. | IC₅₀ (μ/ml) | Hill Slope |
|---|---|---|---|
| ANPH 602 | M-1-018-53-A-fd | 101 | −1.10 |
| Chen pi | M-1-018-54-A-fd | 92 | −1.35 |
| Fang feng | M-1-018-55-A-fd | 261 | −1.48 |
| Bai shao | M-1-018-56-A-fd | 253 | −1.14 |
| Bai zhu | M-1-018-57-A-fd | 311 | −1.21 |
| Yan hu suo | M-1-018-58-A-fd | 18 | −0.75 |
| Wu mei | M-1-018-59-A-fd | 197 | −1.18 |
| Haloperidol* | — | 1.3** | −0.88 |

*positive control
**IC₅₀ in nM

Interaction with Several Additional Receptor Subtypes Implicated in Bowel Disorders

Initial screening (Table 7) indicates that at a concentration of 100 μg/ml, none of the individual herbs in ANPH 602 showed significant interaction with CGRP and Purinergic P_{2Y} receptors. However, Yan Hu inhibited ligand binding to Adrenergic α_{2A}, Histamine H₂, and Sigma σ₁ receptors by 90%, 83%, and 66% respectively.

TABLE 7
Inhibition of ligand binding by individual herbs in
ANPH 602 (100 μg/ml) on various receptors subtypes.

| | Chen pi | Fang feng | Bai shao | Bai zhu | Yan hu suo | Wu mei |
|---|---|---|---|---|---|---|
| | MH-018- | MH-018- | MH-018- | MH-018- | MH-018- | MH-018- |
| | 54-A-fd | 55-A-fd | 56-A-fd | 57-A-fd | 58-A-fd | 59-A-fd |
| Adrenergic α_{2A} | 35 | 18 | 9 | 9 | 90 | 23 |
| CGRP | 9 | 8 | 18 | 25 | 19 | 27 |
| Histamine H₂ | 20 | 25 | 25 | 26 | 83 | 11 |
| Purinergic P_{2Y} | 3 | 12 | 30 | 0 | 19 | 25 |
| Sigma σ₁ | 1 | 0 | 1 | 28 | 66 | 8 |

### Example 2

### HUMAN CLINICAL TRIAL

Based upon the promising pharmacological profiles described above, an open-label, single-arm, fixed-dose, outpatient trial was initiated in China using the ANPH 602 formulation. A description of the trial design, methodology, and current results follows.

The majority of the patients were recruited from an advertisement posted in the XinMin evening news in the Shanghai Municipality, China. Patient screening and subsequent review occurred at the out patient department of the Shu Guang Hospital.

Herbal extracts used were administered within standard dosage levels.

Subjects

Patients between the ages of 18 and 60 (inclusive) were screened by physicians who would be the equivalent of a gastroenterologist in Western medicine. This screening involved a routine clinical work-up for IBS patients with tests as determined appropriate, including a colonoscopy or barium enema. Patients were assessed according to the Rome criteria, a symptom-based classification for IBS, which was developed by a multinational consensus in Rome, 1988. It is an established standard for diagnosis of IBS.

The Rome diagnostic inclusion criteria for IBS include: At least three months of continuous or recurrent symptoms of abdominal pain: characterized by being (i) relieved by defecation, (ii) associated with change in stool frequency, and (iii) associated with change in stool consistency; and two or more of the following: stool frequency: (i) greater than three times a day or less than three times a week; (ii) stool form: lumpy/hard/loose/watery; (iii) stool passage; strain/urgency/ incomplete evacuation; (iv) passage of mucus; (v) bloating/feeling of abdominal distension.

A full list of inclusion and exclusion criteria are as follows.

Inclusion criterion

Patients who meet all of the following inclusion criteria were eligible for enrollment in the study. Age: 18-60, inclusive; History of colonoscopylbarium enema within the previous 5 years; Diagnosis of IBS by Rome criteria; as follows:

At least three months of continuous or recurrent symptoms of abdominal pain: relieved by defecation; associated with change in stool frequency; associated with change in stool consistency.

And two or more of the following: (i) stool frequency greater than three times a day; (ii) stool form: loose/watery; (iii) stool passage urgency, incomplete evacuation; (iv) passage of mucus; (v) bloating/feeling of abdominal distension (vi) Normal liver finction tests and complete blood count and blood urea nitrogen and creatinine levels; (vii) The difference in the BSS score measured at Visit 2 (Baseline) must be ≦20% of the BSS score measured at Visit 1 (Screening).

Exclusion criteria

Patients who met any of the following exclusion criteria are not eligible for enrollment in the study: (i) Constipation predominant IBS; (ii) Current evidence or history of liver disease (iii) Use of any of the following medications, unless the patient is still symptomatic for IBS, and the medications have been used for less than or equal to three months, and the effects of the medications are stable to Anticholinergics, Smooth muscle relaxants, Motility stimulants, and Antidepressants (iv) Current evidence or history (within 24 months of screening) of alcoholism or drug abuse; (v) Current evidence or history of psychiatric illness or dementia; (vi) Current evidence or history of allergies to food additives (vii) Current evidence or history of lactose intolerance; (viii) Current evidence or history of inflammatory bowel disease—regional enteritis, ulcerative colitis, Crohn's disease (ix) Current evidence or history of endometriosis/leimyoma uteri (fibroids); (x) Current evidence or history of diabetes mellitus; (xi) Current evidence of active Schistosomiasis; (xii) Use of Chinese Herbal Medications for IBS within the previous two weeks; (xiii) Patient is pregnant or breast-feeding.

Treatment schedule

After initial gastroenterological screening (Visit 1), all patients enter a two-week baseline phase. A Bowel Symptom Scale (BSS) was completed at the screening visit and again at Visit 2 (14±3 days) later, to assess measurement reliability, and to account for any degree of improvement based simply on admission to the study. Patients received study medication at the end of the baseline phase (Visit 2). The patients were seen at two weeks intervals, ±3 days, (Visit 3 and Visit 4) and then again after 4 weeks, ±3 days (Visit 5). Treatment was administered continuously, involving daily doses for eight weeks. Complete physicals and blood/urine analysis were completed on all patients at the end of the treatment period. Patients were closely monitored for any adverse events or deterioration of symptoms.

Herbal preparation and dispensing

The study medication was administered in a tonic tea formulation. Patients received a total of 13.5 gm/day of medication (one (1) 4.5 gram tonic tea bag, TID) for eight (8) weeks. Compliance was monitored by asking the patients to return all unused packets as well as a record of dispensing.

The Bowel Symptom Scale (BSS)

Only part one (visual analogue portion) of the BSS was administered during the study. The BSS is a validated self-administered instrument for measuring (IBS-specific) health status and outcomes from the patient's point of view. This instrument evaluates pain, distension, bowel dysfunction, and quality of life/global well-being.

Irritable Bowel Syndrome Quality of Life Questionnaire (IBSQOL)

The IBSQOL is a validated self-administered instrument for measuring health status and outcomes (specifically related to IBS) from the patient's point of view. This instrument evaluates emotional functioning, mental health, sleep behavior, energy, physical functioning, diet, social role, physical role, and sexual functioning. The instrument's standardized scoring system yields a profile of nine health scores and a self-evaluated change in health status.

IBS Questionnaire (IBS-Q)

The self-administered instrument is designed to assess relief of IBS pain and discomfort, stool consistency, and average number of bowel movements.

Results

The results for thirty-one patients recruited to the study were available for analysis. Using the criterion of a meaningful response as being at least a 20% decrease in BSS score from baseline, 21 out of 31 patients (68%) have shown a meaningful response/improvement in IBS symptoms. Of these 21 meaningful responders, 15/25 were male and 6/6 were female. Other data of note includes the fact that 29/31 (94%) have noticed some form of numerical improvement from baseline with 2/31 having numerical worsening. Stool consistency averaged 4.02 (1=very hard to 5=watery) at baseline and improved to 3.4 at Week 2. The number of daily bowel movements averaged 2.92 at baseline and decreased to 2.23 after 2 weeks of treatment. These results compare favorably with the active treatment arm in the recent JAMA article. See Bensoussan A. et al. “Treatment of irritable bowel syndrome with Chinese herbal medicine: a randomized controlled trial” JAMA 1998 Nov 11;280(18):1585-9] and exceed the placebo response in that same study.

The various articles of the scientific and/or medical literature, and the U.S. and foreign patents and U.S., foreign and international patent applications cited herein are hereby incorporated by reference. To the extent permissible by law, each constitutes a part of the disclosure of this specification. Furthermore, while specific embodiments, working examples, and/or prophetic examples of the invention have been described in detail to illustrate the broad applicability and principles underlying the invention, it will be understood by those of skill in the art that the invention may be embodied otherwise without departing from such broad applicability and principles. That is to say, although this invention has been described in terms of certain preferred embodiments, other embodiments that are apparent to those of ordinary skill in the art are also within the scope of this invention.

What is claimed is:

1. A composition of matter useful in treating a bowel disorder in a mammal, consisting essentially of:
a pharmaceutically effective amount of the extract “Bai Shao”,
a pharmaceutically effective amount of the extract “Bai Zhu”,
a pharmaceutically effective amount of the extract “Chen Pi”,
a pharmaceutically effective amount of the extract “Fang Feng”;
a pharmaceutically effective amount of the extract “Wu Mei”; and
a pharmaceutically effective amount of the extract “Yan Hu”.

2. A composition of matter useful in treating a bowel disorder in a mammal, consisting essentially of:
a pharmaceutically effective amount of a plant extract selected from the group consisting of “Bai Shao”, “Bai Zhu”, “Chen Pi”, “Fang Feng”, and any combination thereof; and
a pharmaceutically effective amount of a plant extract selected from the group consisting of a “Wu Mei”, and “Yan Hu” and any combination thereof.

3. A composition of matter useful in treating a bowel disorder in a mammal, consisting essentially of:
a pharmaceutically effective amount of the extract “Bai Shao”,
a pharmaceutically effective amount of the extract “Bai Zhu”,
a pharmaceutically effective amount of the extract “Chen Pi”,
a pharmaceutically effective amount of the extract “Fang Feng”; and
a pharmaceutically effective amount of a plant extract selected from the group consisting of a “Wu Mei”, and “Yan Hu” and any combination thereof.

4. A composition of matter useful in treating a bowel disorder in a mammal, consisting essentially of:
a pharmaceutically effective amount of the extract “Bai Shao”,
a pharmaceutically effective amount of the extract “Bai Zhu”,
a pharmaceutically effective amount of the extract “Chen Pi”,
a pharmaceutically effective amount of the extract “Fang Feng”; and
a pharmaceutically effective amount of the plant extract “Wu Mei”.

5. A composition of matter useful in treating a bowel disorder in a mammal, consisting essentially of:
a pharmaceutically effective amount of the extract “Bai Shao”,
a pharmaceutically effective amount of the extract “Bai Zhu”,
a pharmaceutically effective amount of the extract “Chen Pi”,
a pharmaceutically effective amount of the extract “Fang Feng”; and
a pharmaceutically effective amount of the plant extract “Yan Hu”.

6. A composition of matter useful in treating a bowel disorder in a mammal, consisting essentially of:
a pharmaceutically effective amount of the extract “Chen Pi”,
a pharmaceutically effective amount of the plant extract selected from the group consisting of a “Wu Mei”, and “Yan Hu” and any combination thereof.

7. The composition of claims 2, wherein the bowel disorder is selected from the group consisting of Irritable Bowel Syndrome (IBS), Irritable Bowel Disease (IBD), Functional Constipation, Functional Diarrhea, Chronic Functional Abdominal Pain, Non-chronic Functional Abdominal Pain, Functional Abdominal Bloating, Functional Dyspepsia, Functional Heartburn, Non-ulcer Dyspepsia, Non-cardiac Chest Pain, Functional Chest Pain of Presumed Esophageal Origin, Chronic Pelvic Pain, Recurrent Abdominal Pain.

8. The composition of claim 2, wherein the bowel disorder is Irritable Bowel Syndrome.

9. The composition of claim 3, wherein the mammal is a mouse or rat.

10. The composition of claim 2, wherein the mammal is a human.

11. The composition of claim 2, wherein the bowel disorder is treated via a mechanism selected from the group consisting of:
interaction with the CGRP receptor; interaction with a 5HT receptor selected from the group consisting of the 5HT_{1A} receptor subtype, the 5HT₄ receptor subtype, and the 5HT₇ receptor subtype; interaction with a dopamine receptors selected from the group consisting of the dopamine D₁ receptor subtype; the dopamine D₃ receptor subtype, and the dopamine D₅ receptor subtype; and interaction with the sigma 1 (σ1) receptor subtype.

12. The composition of claim 2, wherein the bowel disorder is treated via a mechanism selected from the group consisting of:
inhibition of the CGRP receptor; inhibition of a 5HT receptor selected from the group consisting of the 5HT_{1A} receptor subtype, the 5HT₄ receptor subtype, and the 5HT₇ receptor subtype; inhibition of a dopamine receptors selected from the group consisting of the dopamine D₁ receptor subtype; the dopamine D₃ receptor subtype, and the dopamine D₅ receptor subtype; and inhibition of the sigma 1 (σ1) receptor subtype.

13. The composition of claim 2, wherein the bowel disorder is treated via a mechanism selected from the group consisting of the inhibition of serotonin release, the inhibition of dopamine release, the inhibition of histamine release.

14. The composition of claim 2, wherein the bowel disorder is treated via the mechanism of the inhibition of serotonin.

15. A method of treating a mammal identified as suffering from a bowl disorder, comprising:
administering to the mammal a pharmaceutically effective amount of the composition of claim 2.

16. The method of claim 15, wherein the mammal is a mouse or a rat.

17. The method of claim 15, wherein the mammal is a human.

18. The method of claim 15, wherein the method of administration is oral.

19. The method of claim 15, wherein the bowel disorder is Irritable Bowel Syndrome (IBS).

20. The method of claim 15, wherein the bowel disorder is Inflammatory Bowel Disease (IBD).

21. A method of making a composition for treating one or more symptoms of a bowel disorder, consisting essentially of:
subjecting a first herb selected from the group consisting of “Bai Shao”, “Bai Zhu”, “Chen Pi”, “Fang Feng”, and any combination thereof to a first extraction procedure to yield a first extract;
subjecting a second herb selected from the group consisting of, “Wu Mei” and “Yan Hu”, and any combination thereof to a second extraction procedure to yield a second extract, and
combining the first extract with the second extract to form a composition for treating a bowel disorder.

22. A method of making a composition for treating one or more symptoms of a bowel disorder, comprising:
subjecting a composition consisting essentially of one or more of the following first herbs: “Bai Shao”, “Bai Zhu”, “Chen Pi”, and “Fang Feng”, and one or more of the following second herbs: “Wu Mei” and “Yan Hu”, to an extraction procedure to yield an extract that is a composition for treating a bowel disorder.
